(19) United States
(12) Patent Application Publication
(10) Pub. No.: US 2005/0051922 A1
(43) Pub. Date: Mar. 10, 2005

(54) Pharmaceutical composition with sodium lauryl sulfate as an extra-granular absorption/compression enhancer and the process to make the same

(76) Inventors: Avinash Nangia, Lincoln, RI (US); Boyong Li, Morgantown, WV (US); John R. Cardinal, Tamarac, FL (US)

Correspondence Address: HEDMAN & COSTIGAN P.C., 1185 AVENUE OF THE AMERICAS, NEW YORK, NY, 10036, US

(21) Appl. No.: 10/940,587

(22) Filed: Sep. 14, 2004

Related U.S. Application Data
(63) Continuation-in-part of application No. 10/664,803, filed on Sep. 19, 2003.
(60) Provisional application No. 60/412,180, filed on Sep. 20, 2002.
(60) Provisional application No. 60/412,181, filed on Sep. 20, 2002.

(51) Int. Cl. A61K 9/20; B27N 3/00

(52) U.S. Cl. 264/109; 424/464

(57) ABSTRACT

A process for preparing a pharmaceutical dosage form or core wherein an absorption/compression agent is introduced into the formulation extra-granularly, and a pharmaceutical tablet prepared by said process.

### CROSS REFERENCE TO RELATED APPLICATION

This is a continuation-in-part of U.S. patent application Ser. No. 10/664,803 filed on Sep. 19, 2003 and claims the benefit of provisional patent application Ser. No. 60/412,180 and 60/412,181 filed on Sep. 20, 2002.

### BACKGROUND OF THE INVENTION

The present invention relates to a pharmaceutical unit dose formulation wherein an absorption/compression enhancer is employed extra-granularly. More specifically, the present invention relates to an oral dosage form comprising a water soluble drug, preferably an antihyperglycemic drug such as metformin or buformin, or a pharmaceutically acceptable salt thereof such as metformin hydrochloride or the metformin salts described in U.S. Pat. Nos. 3,957,853 and 4,080,472, which are incorporated herein by reference.

Many techniques have been used in the prior art to provide controlled and extended-release pharmaceutical dosage forms in order to achieve the dual goal of maintaining therapeutic serum levels of medicaments and maximizing patient compliance.

The prior art teaches extended release tablets that have an osmotically active drug core surrounded by a semipermeable membrane. These tablets function by allowing a fluid such as gastric or intestinal fluid to permeate the coating membrane and dissolve the active ingredient, thereby allowing the active ingredient to be released through a passageway in the coating membrane. Alternatively, if the active ingredient is insoluble in the permeating fluid, an expanding agent such as a hydrogel may push it through the passageway. Some representative examples of these osmotic tablet systems can be found in U.S. Pat. Nos. 3,845,770; 3,916,899; 4,034,758; 4,077,407 and 4,783,337. U.S. Pat. No. 3,952,741 teaches an osmotic device wherein the active agent is released from a core surrounded by a semipermeable membrane only after sufficient pressure has developed within the membrane to burst or rupture the membrane at a weak portion of the membrane.

The basic osmotic device described in the above cited patents has been refined over time in an effort to provide greater control over the release of the active ingredient. For example U.S. Pat. Nos. 4,777,049 and 4,851,229 describe an osmotic dosage form comprising a semipermeable wall surrounding a core. The core contains an active ingredient and a modulating agent wherein the modulating agent causes the active ingredient to be released through a passageway in the semipermeable membrane in a pulsed manner. Further refinements have included modifications to the semipermeable membrane surrounding the active core such as varying the proportions of the components that form the membrane, i.e. U.S. Pat. Nos. 5,178,867; 4,587,117 and 4,522,625 or increasing the number of coatings surrounding the active core, i.e., U.S. Pat. Nos. 5,650,170 and 4,892,739.

U.S. Pat. Nos. 6,099,859; 6,284,275; 6,495,162 and U.S. patent application Ser. No. 09/594,637 teach a controlled or sustained release formulation for an antihyperglycemic drug wherein the bioavailability of the drug is not decreased by the presence of food, the dosage form does not employ an expanding polymer, it can provide continuous and non-pulsating therapeutic levels of an antihyperglycemic drug to an animal or human in need of such treatment over a twelve hour to twenty-four hour period and it provides a controlled or sustained release formulation for an antihyperglycemic drug that obtains peak plasma levels approximately 8-12 hours after administration. Furthermore, the osmotic core component, as taught by the above references, may be made using ordinary tablet compression techniques.

Metformin hydrochloride is a brittle drug with high density and poor compressibility. Like other drugs with a brittle fracture nature, it is more sensitive to the rate of compaction, which results in loss of compaction strength, high friability, high weight variability and capping phenomenon.

U.S. Pat. No. 6,117,451 describes using specific excipients with particular size and density to improve the flow and compressibility of metformin hydrochloride. These excipients are blended with metformin and the blend is then directly compressed. The majority of these excipients are of the water-insoluble type and can not be used for systems based on osmotic principles. Additionally, at the level at which these directly compressible materials are used, the size of the finished dosage forms increases significantly.

U.S. Pat. No. 5,955,106 and WP 03/028704A1 describe extended release pharmaceutical compositions with high water content (up to 8%) to aid compression. However, compositions with higher initial moisture content tend to pose serious problems in maintaining the stability of the drug and the release profile, especially in systems based on osmotic principles.

For extended release systems based on osmotic mechanisms, it is critical that the inner drug core remains solid and erodes evenly to maintain the osmotic pressure at saturation. This becomes even more challenging for systems with high drug loading of a highly water-soluble drug, such as metformin. Strong compacts typically allow uniform erosion of the core until the last interval without premature hydration or collapse of the core. If the core collapses prematurely, there is a rapid build up of osmotic pressure within the system, which results in a rapid rate in drug release. Additionally, if the build up of osmotic pressure ruptures in the rate controlling semi-permeable coating it may lead to dose dumping. Since the drug loading in the proposed system is about 90%, there is a need to have a strong core that erodes uniformly inside the system to achieve the desired in vitro dissolution release profile.

Irrespective of the mechanism involved in making the tablet, problems encountered during compression are usually linked to the compact structure. Change from a highly porous mass of discrete particles to one with continuous (but still a porous solid matrix) may play an important role in the tablet's functional characteristics, such as hardness and friability. Since all tablets do not possess a uniform density distribution (i.e. heterogeneous), the nature to greater extent is controlled by the final voidage after initial packing, nature of the material (plastic vs. elastic), its dependency upon the compaction rate and behavior during compression and ejection.

The ability to improve the compressibility of tablets containing water soluble drugs is generally limited to techniques such as wet granulation with a binder or addition of highly compressible fillers or binders. Specifically, metformin formulations require a very high percentage of active ingredients (up to 1000 mg), which leaves minimal room for excipients that can improve the overall compressibility of the solid dosage form, i.e. improved hardness and friability. The formulation taught by U.S. Pat. Nos. 6,099,859; 6,284,275; 6,495,162 and U.S. patent application Ser. No. 09/594,637 employ an absorption enhancer such as sodium lauryl sulfate to improve the bioavailability of metformin. Metformin has previously been shown to have poor absorption in the lower part of the gastrointestinal tract (see Vidon et al., Metformin in the digestive tract, Diabetes Res. Clin. Pract. 4, 223-229, 1988 and Marathe et al. Effect of altered gastric emptying and gastrointestinal motility on bioavalibility of metformin, AAPS Annual Meeting, New Orleans, La. 1999). In addition to being added as an absorption enhancer, sodium lauryl sulfate is also used in formulations as a lubricant to improve flowability of the granulation and reduce ejection force.

It is an object of the present invention to provide a pharmaceutical formulation for a drug using an absorption/compression enhancer added post granulation during the blending stage.

It is an additional object of the present invention to provide a pharmaceutical formulation for a drug that has improved tabletting properties, such as improved tablet hardness, reduced friability, low weight variability and no capping problems.

It is also a further object of the present invention to provide a controlled or sustained release formulation for a drug that can provide continuous and non-pulsating therapeutic levels of the drug to an animal or human in need of such treatment over a twelve hour to twenty-four hour period with improved tablet properties.

It is an additional object of the present invention to provide a controlled or sustained release formulation for a drug that obtains peak plasma levels approximately 8-12 hours after administration with improved tablet properties.

### SUMMARY OF THE INVENTION

The foregoing objectives are met by a process for preparing a tablet dosage form or core comprising the following steps:
(a) preparing a granulation comprising:
   (i) a drug;
   (ii) a binding agent; and
(b) blending the granulation with:
   (i) an absorption/compression enhancer;
   (ii) optionally a lubricant; and
(c) forming a tablet from the blended material.

The above stated process will preferably form an immediate release tablet or a core for a modified release pharmaceutical formulation.

A tablet or core prepared according to the above process may be further coated with a membrane coating wherein the membrane is permeable to the passage of water and biological fluids. The coating should comprise a water insoluble polymer, optionally a flux enhancer and optionally a plasticizer. The coating should also comprise at least one passageway for the release of the drug.

The membrane coated dosage form of the present invention can provide therapeutic levels of the drug for twelve to twenty-four hour periods. In the present invention the absorption/compression enhancer is added during the blending and prior to the compression step as opposed to the granulation steps. The applicant has discovered that this novel approach to the formation of a solid dosage form results in improved compressibility and therefore improved hardness and reduced friability. These improvements in the tablet's hardness and reduced friability increase the tablet's resistance to cracking and splintering caused by tumbling during coating, especially in a fluidized bed coater. Additionally, it was found that the addition of an absorption/compression enhancer after the granulation step reduced variations in tablet weigh and hardness.

To make a strong compact, the particles must move relative to each other to improve the packing density. Lubricants are typically used to achieve this effect. Additionally, lubricants will form a finite continuous coating on the punches and dies. The nature of the lubricant (i.e., hydrophobic vs. hydrophilic), its particles size and shape are critical to its distribution and effectiveness. Hydrophobic lubricants, such as magnesium stearate, calcium stearate and stearic acid, have a laminar structure. They occur as plate-like crystals packed together much like a deck of cards. When blended, the plate-like crystals shear onto adjacent drug or filler particles and evenly coat all surfaces, interrupting bonding sites between the particles surfaces thereby weakening the tablet structure and decreasing hardness. Sodium lauryl sulfate, a hydrophobic surfactant, was used in the formulation as an absorption enhancer to improve the bioavailability of water soluble drugs, such as metformin. When sodium lauryl sulfate was added during the wet granulation of metformin, and the granulation was subsequently lubricated with magnesium stearate, the tablets showed lower hardness and higher friability and weight variability. However, when sodium lauryl sulfate was blended with the granulation during the post-granulation blending step before blending with magnesium stearate, it improved the hardness and friability significantly while eliminating the capping problem completely. When added during the blending stage the angular and asymmetrical shape of the sodium lauryl sulfate coated the hydrophilic drug particles and reduced the interparticulate friction. This improved the free flowing nature of the granulation by reducing the powder bed packing of dense metformin particles, as well as maintaining the pore structure during ejection of the tablets. This also allowed uniform filling of the die cavity with reduced weight variability. By pre-coating the metformin particles with hydrophilic sodium lauryl sulfate particles, the sensitivity of the granulation to over-blending with magnesium stearate also became less critical.

### DETAILED DESCRIPTION OF THE INVENTION

The drug or active pharmaceutical ingredient can be any drug such as those described in Remington: The Science and Practice of Pharmacy (20^{th} Ed. 2000) or the U.S. Pharmacopoeia (26^{th} Ed. 2002), which are incorporated herein by reference. In a preferred embodiment the drug should be water soluble.

Drugs that are very soluble in water and can be used in this invention include prochlorperazine edisylate, ferrous sulfate, amphetamine sulfate, benzphetamine hydrochloride, isoproteronol sulfate, aminocaproic acid, potassium chloride, mecaxylamine hydrochloride, procainamide hydrochloride, methamphetamine hydrochloride, phenmetrazine hydrochloride, bethanechol chloride, methacholine chloride, tridihexethyl chloride, phenformin hydrochloride, methylphenidate hydrochloride, pilocarpine hydrochloride, atropine sulfate, scopolamine bromide, isopropamide iodide, cimetidine hydrochloride, theophylline cholinate, cephalexin hydrochloride, and the like.

The drug can be in various forms, such as uncharged molecules, molecular complexes, pharmacologically acceptable salts such as hydrochloride, hydrobromide, sulfate, laurate, palmitate, tartrate, oleate, phosphate, nitrite, borate, acetate, maleate and salicylate. For acidic drugs, salts of metals, amines or organic cations; for example, quartemary ammonium can be used. Derivatives of drugs such as esters, ethers and amides can also be used. Additionally, a drug that is water insoluble can be used in a form that is a water soluble derivative thereof to serve as a solute, and on its release from the tablet, is converted by enzymes, hydrolyzed by body pH or other metabolic processes to the original biologically active form.

Examples of other drugs that can be delivered by this invention include aspirin, indomethacin, naproxen, imipramine, levodopa, chloropromazine, methyldopa, dihydroxyphenylalanine, nitroglycerin, isosorbide dinitrate, propranolol, timolol, atenolol, alprenolol, cimetidine, fenoprofen, sulindac, indoprofen, clonidine, pivaloyloxyethyl ester of alpha-methyldopa hydrochloride, theophylline, mefenamic, flufenamic, difuninal, nimodipine, nitrendipine, nisoldipine, nicardipine, felodipine, lidoflazine, tiapamil, gallopamil, amlodipine, mioflazine, calcium gluconate, ketoprofen, ibuprofen, cephalexin, erythromycin, quanbenz, hydrochlorothiazide, ranitidine, flurbiprofen, fenbufen, fluprofen, tolmetin, haloperidol, zomepirac, chlordiazepoxide hydrochloride, diazepam, amitriptylin hydrochloride, imipramine hydrochloride, imipramine pamoate, captopril, ramipril, endlapriat, famotidine, nizatidine, sucralfate, ferrous lactate, vincamine, phenoxybenzamine, diltiazem, milrinone, captopril, madol, alolofenac, lisinolpril, enalapril, etintidine, tertatolol, minoxidil, chlordiazepoxide and the like.

Examples of other relatively soluble drugs which may be included in the formulations of the present invention include vasodilators (e.g., papaverine, diltiazem), cholinergics (e.g., neostigmine, pyridostigmine), antihistamines (e.g., dimenhydrinate, diphenhydramine, chlorpheniramine and dexchlorpheniramine maleate), non-steroidal anti-inflammatory agents (e.g., naproxen, diclofenac, ibuprofen, aspirin, sulindac), gastrointestinals and anti-emetics (e.g., metoclopramide), analgesics (e.g., aspirin, codeine, morphine, dihydromorphone, oxycodone, etc.), anti-epileptics (e.g., phenyloin, meprobamate and nitrezepam), anti-tussive agents and expectorants (e.g., codeine phosphate), antituberculosis agents (e.g., isoniazid), anti-spasmodics (e.g. atropine, scopolamine), diuretics (e.g., bendrofluazide), anti-hypertensives (e.g., propranolol, clonidine), bronchodilators (e.g., albuterol), laxatives, antacids, vitamins (e.g., ascorbic acid), sympathomimetics (e.g., ephedrine, phenylpropanolamine), iron preparations (e.g., ferrous gluconate), anti-muscarinics (e.g., anisotropine), hormones (e.g., insulin, heparin), anti-inflammatory steroids (e.g., hydrocortisone, triamcinolone, prednisone), antibiotics (e.g., penicillin v, tetracycline, clindamycin, novobiocin, metronidazde, cloxacillin), antihemorrhoidals, antidiarrheals, mucolytics, sedatives and decongestants. The above list is not exhaustive.

In an alternative embodiment of the present invention, the drug employed in the core is an antihyperglycemic drug. The term antihyperglycemic drug, as used in this specification, refers to drugs that are useful in controlling or managing noninsulin-dependent diabetes mellitus (NIDDM). Preferably, the antihyperglycemic drug is a biguanide such as metformin or buformin or a pharmaceutically acceptable salt thereof such as metformin hydrochloride.

In addition to the drug, the core, which comprises the granules and the absorption/compression enhancer, should further comprise at least one pharmaceutical excipient such as a binder, plasticizer, diluent, flow aid, lubricant, osmopolymer, osmagen and combinations of the foregoing. These excipients, if used, can be added at the granulation stage or mixed with the granules prior to, along with or subsequent to the addition of the absorption/compression enhancer.

The binding agent may be any conventionally known pharmaceutically acceptable binder such as polyvinyl pyrrolidone, hydroxypropyl cellulose, hydroxyethyl cellulose, ethylcellulose, polymethacrylate, waxes and the like. Mixtures of the aforementioned binding agents may also be used. Preferred binding agents are water soluble, such as polyvinyl pyrrolidone, which has an average molecular weight of 25,000 to 3,000,000. Polyvinyl pyrrolidone is commercially available as POVIDONE® K90. If a binding agent is used it should comprise approximately about 0% to about 40% of the total weight of the core and preferably about 3% to about 15% of the total weight of the core.

The absorption/compression enhancer can be selected from excipients such as a fatty acid, a surfactant, a chelating agent, a bile salt or mixtures thereof. Examples of some preferred absorption/compression enhancers are fatty acids such as capric acid, oleic acid and their monoglycerides; surfactants such as sodium lauryl sulfate, sodium taurocholate and polysorbate 80; and chelating agents such as citric acid, phytic acid, ethylenediamine tetraacetic acid (EDTA) and ethylene glycol-bis (P-aminoethyl ether)-N,N,N,N-tetraacetic acid (EGTA). The absorption/compression enhancer should comprise approximately 0.1% to about 20 of the tablet weight of the core and most preferably about 1% to about 10% of the total weigh of the core.

It has been found that the compressibility of a metformin composition was greatly enhanced by adding sodium lauryl sulfate as the absorption/compression enhancer in a concentration as low 1-5%, preferably around 2.5%, during the blending step, i.e. after the granulation step. This resulted in an increase in the hardness of the tablet from about 10 kp to about 25 kp (see Examples III-VI).

The core may also contain a water soluble diluent or filler. The diluent may be any conventionally known pharmaceutically acceptable diluent, such as lactose, dextrose, sucrose, sodium chloride, maltose, fructose, galactose, gelatin, polyvinylpyrrolidone, rice starch, corn starch, calcium carbonate and the like or mixtures thereof. If a diluent is used in the core it should comprise approximately 0% to about 75% of the total weight of the core and preferably about 2% to about 50% of the total weight of the core.

Suitable lubricants which can be used in preparing compressed forms of the present invention may include talc, stearic acid, magnesium stearate, glyceryl monostearate, glyceryl stearate, sodium stearyl fumerate, hydrogenated oils, polyethylene glycols, glyceryl behenate and sodium stearate.

Suitable flow aids which can also be used in the present invention may include talc, silicon dioxide (which is sold under the tradename AEROSIL® by Degussa) and metallic stearates.

The core may also contain an osmopolymer. Osmopolymers interact with water and aqueous biological fluids and swell or expand to an equilibrium state. Osmopolymers exhibit the ability to swell in water and to retain a significant portion of the imbibed and absorbed water within a polymer structure. Suitable osmopolymers include, but are not limited to, hydroxypropyl methylcellulose, alkylcellulose, hydroxyalkylcellulose, poly(alkylene oxide), or combinations thereof. Other examples of osmopolymers are provided in U.S. Pat. Nos. 4,612,008; 4,327,725; and 5,082,668; which are incorporated herein by reference. An osmopolymer can also function as a binding agent for the core.

The core may also contain an osmagen. An osmagen is a material which attracts fluid into the core of a pharmaceutical tablet. Materials which may be suitable as osmagens include electrolytes and organic acids. Example of useful materials include simple sugars, such as lactose and sucrose, salts such as magnesium sulfate, potassium chloride, ammonium chloride, calcium sulfate, sodium chloride, calcium lactate, mannitol, urea, inositol, magnesium succinate, lithium chloride, lithium sulfate, potassium sulfate, sodium carbonate, sodium sulfate, potassium acid phosphate, tartaric acid, citric acid, itaconic acid, fumaric acid, lactic acid, ascorbic acid, malic acid, maleic acid and the like or combinations thereof. Other osmagens are described in U.S. Pat. Nos. 4,612,008; 5,082,668 and 5,916,596; which are incorporated herein by reference.

In a preferred embodiment of the present invention, the core comprises an antihyperglycemic drug, a binder, an absorption/compression enhancer and a lubricant. The core is preferably formed by wet granulating a drug and a binder followed by blending the granules with an absorption/compression enhancer and a lubricant, and finally compressing the blend into a tablet on a rotary press. The core may also be formed by dry granulating a drug and a binder followed by blending the granules with an absorption/compression enhancer and a lubricant followed by compression into tablets.

The core may optionally be coated with a seal coat, preferably a water-soluble seal coat, such as OPADRY® Clear. The seal coat is used to protect the core during the remainder of the tabletting processing. OPADRY® is a coating system which combines polymers, plasticizers and, if desired, pigments. The seal coat may also comprise an osmotic agent or osmagen such as the sodium chloride described above.

The seal coated core is further coated with a membrane, preferably a modified polymeric membrane to form the controlled or sustained release tablet of the present invention. The membrane is permeable to the passage of external fluids such as water and biological fluids and comprises a film forming polymer, preferably a film forming water insoluble polymer and most preferably a water insoluble cellulose derivative. Additionally, the membrane is impermeable to the passage of the drug in the core. Water insoluble polymers that are useful in forming the membrane are cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate and cellulose acetate butyrate. Other suitable polymers are described in U.S. Pat. Nos. 3,845,770; 3,916,899; 4,008,719; 4,036,228 and 4,612,008; which are incorporated herein by reference. The most preferred water insoluble polymer is cellulose acetate, which comprises an acetyl content of 39.3% to 40.3%. This product is commercially available from Eastman Fine Chemicals.

The membrane can be formed using the above-described water insoluble polymers in combination with a flux enhancing agent. The flux enhancing agent increases the volume of fluid imbibed into the core to enable the dosage form to dispense substantially all of the drug through the passageway and/or the porous membrane. The flux enhancing agent can be a water soluble material or an enteric material. Some examples of the preferred materials that are useful as flux enhancers are sodium chloride, potassium chloride, sucrose, sorbitol, poloxamers (available as PLURONIC® F-68 and PLURONIC® F-127), mannitol, polyethylene glycol (PEG), propylene glycol, hydroxypropyl cellulose, hydroxypropyl methycellulose, hydroxypropyl methycellulose phthalate, cellulose acetate phthalate, polyvinyl alcohols, methacrylic acid copolymers and mixtures thereof. In the preferred embodiment of the invention the flux enhancer is polyethylene glycol 400.

The membrane may also be formed with other commonly known excipients such as plasticizers. Some commonly known plasticizers include adipate, azelate, enzoate, citrate, stearate, isoebucate, sebacate, triethyl citrate, tri-n-butyl citrate, acetyl tri-n-butyl citrate, citric acid esters and those described in the Encyclopedia of Polymer Science and Technology, Vol. 10 (1969), published by John Wiley & Sons. The preferred plasticizers are triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, poloxamers (available as PLURONIC® F-68 and PLURONIC® F-127), triethylcitrate, tributylcitrate, glyceroltributyrate and the like. Depending on the particular plasticizer, amounts from 0% to about 25%, and preferably about 2% to about 15% of the plasticizer can be used based upon the total weight of the coating. The preferred plasticizer is triacetin.

As used herein the term passageway includes an aperture, orifice, bore, hole, weakened area or an erodible element such as a gelatin plug that erodes to form an osmotic passageway for the release of the antihyperglycemic drug from the dosage form. A detailed description of a sustained release coating passageways can be found in U.S. Pat. Nos. 3,845,770; 3,916,899; 4,034,758; 4,077,407; 4,783,337 and 5,071,607.

Generally, the membrane coating around the core will comprise from about 1% to about 5% and preferably about 2% to about 3% based on the total weight of the core and the coating.

In an alternative embodiment, the dosage form of the present invention may also comprise an effective amount of a drug that is available for immediate release. The effective amount of drug for immediate release may be coated onto the membrane of the dosage form or it may be incorporated into the membrane.

In a preferred embodiment the dosage form will have the following composition:

| | Preferred | Most Preferred |
|---|---|---|
| CORE: | | |
| drug | 50-98% | 75-95% |
| binder | 0-40% | 3-15% |
| absorption/compression | | |
| enhancer | 0.1-20% | 1-10% |
| lubricant | 0-10% | 0-5% |
| SEMI-PERMEABLE MEMBRANE: | | |
| Film forming polymer | 50-99% | 75-95% |
| flux enhancer | 0-40% | 2-20% |
| plasticizer | 0-25% | 2-15% |

### DESCRIPTION OF THE PREFERRED EMBODIMENTS

Metformin hydrochloride tablets in accordance with the present invention were prepared as follows. The following experiments demonstrates the improved hardness and other advancements resulting from the addition of an absorption/compression enhancer after the granulation step (extra-granular) in relation to a dosage form wherein the absorption/compression enhancer is added during the granulation step (intra-granular).

### EXAMPLE I

A pharmaceutical extended-release tablet of metformin HCl is prepared as follows:

A. Granulation

139.94 kg of metformin HCl is delumped by passing it through a Comil equipped with a #813 screen and granulated in a Glatt GPCG-60 fluid bed coater with a 32″ Wurster column by spraying 10.06 kg of Povidone K-90 solution in 191.19 kg of purified water (bottom spray) at a spraying rate of 500-1200 g/min, a product temperature of 38-43° C. and an atomization air pressure of 2.5-3 bars. The granules are then discharged and sized through a Comil equipped with a #1143 screen.

B. Blending and Compression

149.89 kg of metformin HCl granules are blended with 7.228 kg of sodium lauryl sulfate in a 20-ft³ slant-cone blender and then blended with 0.790 kg of magnesium stearate. The blend is then compressed into tablets weighing approximately 1129 mg on a 32-station tablet press equipped with ½″ tooling.

C. Seal Coating

56.62 kg of the uncoated tablets are then seal coated in a 36″ coating pan with 2.356 kg of OPADRY4 Clear solution in 21.20 kg of purified water at an exhaust temperature of 40-47° C., an atomization air pressure of 40 psi and a spray rate of 130-180 g/min.

D. Semi-Permeable Membrane Coating

59.07 kg of seal coated tablets are then coated in a Glatt GPCG-60 fluid-bed coater with an 18″ Wurster column with a solution comprising 0.792 kg of cellulose acetate, 0.046 kg of Triacetin, USP, 0.093 kg of Polyethylene Glycol 400, NF in 31.10 kg of Acetone, NF at a product temperature of 20-25° C., a spray rate of about 300 g/min and an atomization air pressure of about 2 bars.

E. Laser Drilling

The membrane coated tablets are then drilled to form one 0.5 mm orifice on each side of the tablets using a Duplex Laser Tablet Driller.

### EXAMPLE II

A pharmaceutical extended-release tablet of metformin HCl is prepared as follows:

A. Granulation

139.14 kg of metformin HCl is delumped by passing it through a Comil equipped with a #813 screen and granulated in a Glatt GPCG-60 fluid bed coater with a 32″ Wurster column by spraying 10.86 kg of Povidone K-90 solution in 206.34 kg of purified water (bottom spray) at a spraying rate of 500-1200 g/min, a product temperature of 38-43° C. and an atomization air pressure of 2.5-3 bars. The granules are then discharged and sized through a Comil equipped with a #1143 screen.

B. Blending and Compression

299.19 kg of metformin HCl granules are blended with 14.34 kg of sodium lauryl sulfate in a 20-ft³ slant-cone blender and then blended with 1.576 kg of magnesium stearate. The blend is then compressed into tablets weighing approximately 1129 mg on a 32-station tablet press equipped with ½″ tooling.

C. Seal Coating

60 kg of the uncoated tablets are then seal coated in a 36″ coating pan with a solution comprising 2.49 kg of OPADRY® Clear in 22.39 kg of purified water at an exhaust temperature of 40-47° C., an atomization air pressure of 40 psi and a spray rate of 130-180 g/min.

D. Semi-Permeable Membrane Coating

61.488 kg of seal coated tablets are then coated in a Glatt GPCG-60 fluid-bed coater with an 18″ Wurster column with a solution comprising 2.451 kg of cellulose acetate, 0.145 kg of Triacetin, and 0.289 kg of polyethylene glycol in 54.80 kg of acetone at a product temperature of 20-25° C., a spray rate of about 300 g/min and an atomization air pressure of about 2 bars.

E. Laser Drilling

The membrane film coated tablets are then drilled to form one 0.5 mm orifice on each side of the tablets using a Duplex Laser Tablet Driller.

F. Color Coating

The laser drilled tablets are then coated in a 36″ coating pan with an OPADRY® White suspension in water at production temperatures of 40-46° C., a spray rate of 120-240 g/min and an atomization air pressure of 40-60 psi.

### EXAMPLE III

A solid dosage form comprising metformin not in accordance with the present invention was produced with sodium lauryl sulfate added intra-granularly.

13.35 kg of metformin HCl was blended with 0.69 kg of sodium lauryl sulfate and then granulated in Glatt GPCG-15 granulators by spraying a binder solution consisting of 0.96 kg of Povidone K-90 previously dissolved in 18.24 kg of purified water, USP. 2.80 kg of the granules were then blended with 0.014 kg of magnesium stearate. The blend was compressed on a sixteen-station tablet press with a ½″ standard concave tooling. The resulting hardness of the tablets prepared as described above was 8.9 kp.

### EXAMPLE IV

A solid dosage form comprising metformin in accordance with the present invention was produced with sodium lauryl sulfate added extra-granularly.

14.04 kg of metformin HCl was granulated in a Glatt GPCG-15 granulator by spraying a binder solution consisting of 0.96 kg of Povidone K-90 previously dissolved in 18.24 kg of purified water, USP onto said metformin HCl. 2.80 kg of the granules were then blended without sodium lauryl sulfate, followed by blending with 0.014 kg of magnesium stearate. Finally, the blends were compressed on a sixteen-station tablet press with a ½″ standard concave tooling. The resulting hardness of the tablet prepared as described above was 10.5 kp.

### EXAMPLE V

A solid dosage form comprising metformin in accordance with the present invention was produced with sodium lauryl sulfate added extra-granularly.

14.04 kg of metformin HCl was granulated in a Glatt GPCG-15 granulator by spraying a binder solution consisting of 0.96 kg of Povidone K-90 previously dissolved in 18.24 kg of purified water, USP onto said metformin HCl. 2.671 kg of the granules were then blended with 0.129 kg of sodium lauryl sulfate and with 0.014 kg of magnesium stearate. Finally, the blends were compressed on a sixteen-station tablet press with a ½″ standard concave tooling. The resulting hardness of the tablet prepared as described above was 26.8 kp.

### EXAMPLE VI

A solid dosage form comprising metformin in accordance with the present invention was produced with sodium lauryl sulfate added extra-granularly.

14.04 kg of metformin HCl was granulated in a Glatt GPCG-15 granulator by spraying a binder solution consisting of 0.96 kg of Povidone K-90 previously dissolved in 18.24 kg of purified water, USP onto said metformin HCl. 0.9725 kg of the granules were then blended with 0.0250 kg of sodium lauryl sulfate and with 0.0025 kg of magnesium stearate. Finally, the blends were compressed on a sixteen-station tablet press with a 12″ standard concave tooling. The resulting hardness of the tablet prepared as described above was 25.6 kp.

The tablets of Examples III-VI were prepared using conditions similar to those described in steps A and B of Example I.

As can be seen by comparing Example III with Examples IV-VI, when the sodium lauryl sulfate is added intra-granularly the hardness of the tablets is lower than when the sodium lauryl sulfate is added extra-granularly. Also as the percentage of sodium lauryl sulfate in the extra-granular blending stage is increased from 0% to 0.25% to 0.50% the hardness of the tablet increased from 10.5 kp to 26.8 kp to 25.6 kp.

### EXAMPLE VII

A solid dosage form comprising metformin was prepared in accordance with the present invention using conditions similar to steps A and B of Example I. Specifically, a 500.00 mg tablet of metformin HCl was prepared in a Glatt GPCG-15 granulator by spraying a binder solution consisting of Povidone K-90 onto metformin HCl and sodium lauryl sulfate. The granules were then blended with magnesium stearate, the blend comprising 561.80 mg of the granules and 2.82 mg of magnesium stearate. Finally, the blend was compressed into 564.62 mg core tablet on a sixteen-station tablet press with a ½″ standard concave tooling.

### EXAMPLE VIII

A solid dosage form comprising metformin was prepared in accordance with the present invention using conditions similar to steps A and B of Example I. Specifically, a 500.00 mg tablet of metformin HCl was prepared in a Glatt GPCG-15 granulator by spraying a binder solution consisting of 35.96 mg of Povidone K-90 onto 500.00 mg metformin HCl. The granules were then blended with sodium lauryl sulfate and magnesium stearate, the blend comprising 535.96 mg of granules, 25.84 mg of sodium lauryl sulfate and 2.82 mg of magnesium stearate. Finally, the blend was compressed into a 564.82 mg core tablet on a sixteen-station tablet press with a ½″ standard concave tooling.

The tablets prepared in Example VIII exhibited a hardness of 16.67 kp (±1.8) versus 5.7 kp (±0.9) for the tablets prepared in Example VII. Additionally, as shown by the results in Table I, there was less variation in tablet weight and hardness of the tablets. The friability percentage (number of chipped or broken tablets) was lowered from 0.2% to 0.03%. Tests showing edge chipping after the friability test, openings on the edge of the tablet after film coating in a fluidized-bed coater, and minor defects on the edge of the tablet after semi-permeable film coating, all showed improvements in the extra-granular tablets versus the intra-granular tablets.

### EXAMPLE IX

A solid dosage form comprising metformin was prepared in accordance with the present invention using conditions similar to steps A and B of Example I. Specifically, a 1000.00 mg tablet of metformin HCl was prepared in a Glatt GPCG-15 granulator by spraying a binder solution consisting of 71.91 mg of Povidone K-90 onto 1000 mg of metformin HCl and 51.69 mg of sodium lauryl sulfate. The granules were then blended with 5.65 mg of magnesium stearate. Finally, the blend was compressed into 1129.25 mg core tablets on a sixteen-station tablet press with a ½″ standard concave tooling.

### EXAMPLE X

A solid dosage form comprising metformin was prepared in accordance with the present invention using conditions similar to steps A and B of Example I. Specifically, a 1000.00 mg tablet of metformin HCl was prepared in a Glatt GPCG-15 granulator by spraying a binder solution consisting of 71.91 mg of Povidone K-90 onto 1000 mg metformin HCl. The granules were then blended with 51.69 mg of sodium lauryl sulfate and 5.65 mg of magnesium stearate. Finally, the blend was compressed into 1129.25 mg core tablets on a sixteen-station tablet press with a ½″ standard concave tooling.

The tablets prepared in Example X exhibited a hardness of 29.1 kp (±2.8) versus 12.8 kp (±2.6) for the tablets prepared in Example IX. Additionally, as shown by the results in Table I, there was less variation in tablet weight and hardness of the tablets. The friability percentage (number of chipped or broken tablets) was lowered from 0.2% to 0.06%. Tests showing edge chipping after the friability test, openings on the edge of the tablet after film coating in a fluidized-bed coater, and minor defects on the edge of the tablet after semi-permeable film coating, all showed improvements in the extra-granular tablets versus the intra-granular tablets.

For a detailed analysis of the data described in Examples VII-X see the following table:

TABLE I
Unit Dose Composition and Performance of Metformin HCl Tablets,
500 mg and 1000 mg, with Sodium Lauryl Sulfate added
Intra-Granularly vs. Extra-granularly

| | Unit Composition (mg/tablet) | | | |
|---|---|---|---|---|
| | EXAM- | EXAM- | EXAM- | EXAM- |
| Components | PLE VII | PLE VIII | PLE IX | PLE X |
| Granules: | | | | |
| Metformin | 500.00 | 500.00 | 1000.00 | 1000.00 |
| Hydrochloride, BP | | | | |
| Sodium Lauryl Sulfate, | 25.84 | — | 51.69 | — |
| NF | | | | |
| Povidone K90, USP | 35.96 | 35.96 | 71.91 | 71.91 |
| Subtotal: | 561.80 | 535.96 | 1123.60 | 1071.91 |
| Tablets: | | | | |
| Metformin HCl Granules | 561.80 | 535.96 | 1123.60 | 1071.91 |
| Sodium Lauryl Sulfate, | — | 25.84 | — | 51.69 |
| NF | | | | |
| Magnesium Stearate, NF | 2.82 | 2.82 | 5.65 | 5.65 |
| Total | 564.62 | 564.62 | 1129.25 | 1129.25 |
| Parameters | Performance | | | |
| Hardness ± SD (kp) | 5.7 ± 0.9 | 16.7 ± 1.8 | 12.8 ± 2.6 | 29.1 ± 2.8 |
| Variation in hardness | 15.5 | 11.0 | 20.0 | 9.8 |
| (% RSD) | | | | |
| Tablet Weight Variation | 1.9 | 0.6 | 2.6 | 0.54 |
| (% RSD) | | | | |
| Friability, % | 0.2 | 0.03 | 0.2 | 0.06 |
| Edge chipping after | major | medium | major | minor |
| friability test¹ | | | | |
| Opening on edge after | — | none | 6% | 0% |
| film coating in fluid-bed | | | | |
| Minor defects on edge | — | none | 9% | 3% |
| after film coating | | | | |

¹Edge chipping grade:
Major-extensive and deep chipping;
Medium-about ⅓ to ⅓ edge chipping and less deep;
Minor-a few shallow chips.

As can be seen above, the hardness of the tablets increased from 5.7±0.9 kp to 16.7±1.8 kp for the 500 mg tablet and from 12.8±2.6 kp to 29.1±2.8 kp for the 1000 mg tablet when the absorption/compression enhancer, herein sodium lauryl sulfate, was added extra-granularly. In addition, improvements have been made to the tablet weight variation, edge chipping, edge openings and minor defects after applying the sustained release membrane coating in the fluid bed coater.

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

1. A process for preparing a pharmaceutical dosage form comprising the following steps:
(a) granulating:
(i) a drug; and
(ii) at least one pharmaceutically acceptable excipient;
(b) blending the granules prepared in step (a) with an absorption/compression enhancer; and optionally a lubricant; and
(c) compressing the blended material from step (b) into a tablet.

2. A process as defined in claim 1 further comprising the step of applying a seal coat to said tablet prepared in step (c).

3. A process as defined in claim 1 further comprising the step of applying a membrane coating to said tablet prepared in step (c).

4. A process as defined in claim 3 further comprising the step of forming a passageway in said membrane coating.

5. A process as defined in claim 1 further comprising the steps of
(d) applying a seal coat to said tablet prepared in step (c);
(e) applying a membrane coating to the seal coated tablet of step (d) and
(f) forming a passageway in said membrane.

6. A process as defined in claim 1 wherein said drug is water soluble.

7. A process as defined in claim 1 wherein said drug is an antihyperglycemic drug.

8. A process as defined in claim 7 wherein said antihyperglycemic drug is metformin or a pharmaceutically acceptable salt thereof.

9. A process as defined in claim 7 wherein said antihyperglycemic drug is buformin or a pharmaceutically acceptable salt thereof.

10. A process as defined in claim 1 wherein said pharmaceutical excipient is a water soluble binding agent.

11. A process as defined in claim 10 wherein said water soluble binding agent is selected from the group consisting of polyvinyl pyrrolidone, hydroxypropyl cellulose, hydroxyethyl cellulose, waxes or mixtures thereof.

12. A process as defined in claim 1 wherein said pharmaceutical excipient is an absorption/compression enhancer selected from the group consisting of fatty acids, surfactants, chelating agents, bile salts or mixtures thereof.

13. A process as defined in claim 3 wherein said membrane coating is a water insoluble cellulose derivative.

14. A process as defined in claim 13 wherein said water insoluble cellulose derivative is cellulose acetate.

15. A process as defined in claim 3 wherein said membrane coating further comprises a plasticizer and a flux enhancer.

16. A process as defined in claim 15 wherein said flux enhancer is selected from the group consisting of sodium chloride, potassium chloride, sucrose, sorbitol, mannitol, polyethylene glycol, propylene glycol, hydroxypropyl cellulose, hydroxypropyl methycellulose, poloxamers, hydroxypropyl methycellulose phthalate, cellulose acetate phthalate, polyvinyl alcohols, methacrylic acid copolymers or mixtures thereof.

17. A process as defined in claim 16 wherein said plasticizer is selected from the group consisting of triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, poloxamers, triethylcitrate, tributylcitrate, glyceroltributyrate and mixtures thereof.

18. A process as defined in claim 17 wherein said plasticizer is triacetin.

19. A process as defined in claim 4 wherein at least two passageways are formed in the membrane coating.

20. A solid dosage form prepared according to claim 1.

21. A pharmaceutical dosage form, prepared by:
(a) granulating a drug; and at least one pharmaceutically acceptable excipient;
(b) blending the granules of step (a) with an absorption/compression enhancer and optionally a lubricant; and
(c) compressing the blended material from step (b) into a tablet.

22. A pharmaceutical dosage form, as defined in claim 21, further comprising a seal coat.

23. A pharmaceutical dosage form, as defined in claim 21, further comprising a membrane coating covering said tablet.

24. A pharmaceutical dosage form as defined in claim 23 wherein said membrane comprises a water insoluble cellulose derivative.

25. A pharmaceutical dosage form as defined in claim 23 wherein said tablet comprises 75-95% of an antihyperglycemic drug; 3-15% of a binding agent; 1-10% of an absorption/compression enhancer and 0-10% of a lubricant; and said membrane coating covering said tablet comprises 75-95% of a film forming water insoluble polymer; 2-20% of a flux enhancer and 2-15% of a plasticizer; and further comprises at least one passageway in said membrane for release of said antihyperglycemic drug.

26. A pharmaceutical dosage form, as defined in claim 22, further comprising a membrane coating covering said tablet.

27. A pharmaceutical dosage form, as defined in claim 26, wherein said membrane coating further comprises a film forming water insoluble polymer.

28. A pharmaceutical dosage form, as defined in claim 27 wherein said membrane coating further comprises a flux enhancer.

29. A pharmaceutical dosage form, as defined in claim 28 wherein said membrane coating further comprises a plasticizer.

30. A pharmaceutical dosage form, as defined in claim 29 wherein said membrane coating further comprises at least one passageway in said membrane coating for release of said drug.

31. A pharmaceutical dosage form, as defined in claim 21, wherein said granules comprise an antihyperglycemic drug and a binding agent, said tablet comprising 50-98% by weight of said tablet of said antihyperglycemic drug; 0-40% by weight of said tablet of said binding agent; 0.1-20% by weight of said tablet of said absorption/compression enhancer and 0-20% by weight of said tablet of said lubricant.

32. A pharmaceutical dosage form as defined in claim 21 wherein said drug is water soluble.

33. A pharmaceutical dosage form as defined in claim 21 wherein said drug is an antihyperglycemic drug.

34. A pharmaceutical dosage form as defined in claim 31 wherein said antihyperglycemic drug is metformin or a pharmaceutically acceptable salt thereof.

35. A pharmaceutical dosage form as defined in claim 33 wherein said antihyperglycemic drug is buformin or a pharmaceutically acceptable salt thereof.

36. A pharmaceutical dosage form as defined in claim 31 wherein said binding agent is a water soluble binding agent.

37. A pharmaceutical dosage form as defined in claim 31 wherein said binding agent is selected from the group consisting of polyvinyl pyrrolidone, hydroxypropyl cellulose, hydroxyethyl cellulose, waxes or mixtures thereof.

38. A pharmaceutical dosage form as defined in claim 27 wherein said water insoluble polymer is a cellulose derivative.

39. A pharmaceutical dosage form as defined in claim 31 wherein at least two passageways are formed in the membrane.

40. A pharmaceutical dosage form consisting essentially of a tablet prepared by
(a) forming granules consisting essentially of:
(i) metformin or a pharmaceutically acceptable salt thereof; and
(ii) a binding agent;
(b) blending said granules with an absorption/compression enhancer and a lubricant;
(c) surrounding said tablet with a seal coat;
(c) covering said seal coated tablet with a membrane coating consisting of:
(i) a film forming water insoluble cellulose derivative;
(ii) a plasticizer;
(iii) a flux enhancer; and
(d) forming at least one passageway in the membrane.

41. A pharmaceutical dosage form, according to claim 40 wherein said tablet consists essentially of 75-95% of metformin hydrochloride; 3-15% of said binding agent; 2-15% of said absorption/compression enhancer; 0-10% of said lubricant; and said membrane coating consists essentially of 75-95% of said water insoluble cellulose derivative; 2-20% of said plasticizer; 2-15% of said flux enhancer; and further comprising at least one passageway in the membrane for the release of the antihyperglycemic drug.

42. A pharmaceutical dosage form, according to claim 41 wherein said absorption/compression enhancer is sodium lauryl sulfate.
